(19) United States
(12) Patent Application Publication
(10) Pub. No.: US 2026/0193554 A1
(43) Pub. Date: Jul. 9, 2026

(54) PRODUCTION OF SYNTHETIC FUELS FROM CARBON DIOXIDE WITH CARBON DIOXIDE SEPARATION

(71) Applicant: IFP Energies Nouvelles, Rueil-Malmaison (FR)

(72) Inventors: Catherine LAROCHE, Rueil-Malmaison Cedex (FR); Jean-Francois JOLY, Rueil-Malmaison Cedex (FR); Marie DEHLINGER, Rueil-Malmaison Cedex (FR); Jean-Philippe HERAUD, Rueil-Malmaison Cedex (FR)

(73) Assignee: IFP Energies Nouvelles, Rueil-Malmaison (FR)

(21) Appl. No.: 19/132,416

(22) PCT Filed: Nov. 20, 2023
(86) PCT No.: PCT/EP2023/082356

§ 371 Date: May 23, 2025

(30) Foreign Application Priority Data
Nov. 25, 2022 (FR) FR2212353

(51) Int. Cl. C10G 69/02; B01D 53/02; B01D 53/14; B01J 19/00; B01J 19/24; C01B 32/40; C07C 1/04; C10G 3/00; C25B 1/04; C25B 15/08

(52) U.S. Cl. CPC C10G 69/02; B01D 53/02; B01D 53/1475; B01J 19/0013; B01J 19/245; C01B 32/40; C07C 1/0415; C07C 1/0435; C07C 1/044; C07C 1/048; C10G 3/45; C25B 1/04; C25B 15/081; B01D 2257/504; B01D 2258/0283; B01J 2219/00087; B01J 2219/00157; C07C 2523/745; C07C 2523/75; C10G 2300/4043; C10G 2300/4081

(57) ABSTRACT

Device/process for capturing/converting CO₂, comprising/using a unit (2) for CO₂ capture from the feedstock (1) that produces a CO₂-rich effluent (3), a water electrolysis unit (5) that converts water (4) into oxygen (6) and hydrogen (7), an RWGS reaction unit (8) that treats the CO₂-rich effluent with the hydrogen and produces an RWGS gas (9) enriched in CO and in water, an FT reaction unit (13) that converts the RWGS gas and produces an FT effluent (14), a first separation unit (15) that treats the FT effluent and produces a hydrocarbon effluent (17) and a gaseous effluent (33), a second separation unit (34) that separates a first gas (33), producing a CO₂-depleted gas (18), and sends a CO₂-rich gas (35) to the RWGS unit, a hydrogen reaction unit (20) that treats the hydrocarbon effluent in order to produce hydrocarbon cuts (21).

### TECHNICAL FIELD

The present invention relates to the production of synthetic fuels, namely gasoline, kerosene, gas oil, and/or other hydrocarbon products, such as naphtha, or lubricant bases, of very high quality (essentially free of sulfur, aromatics, nitrogen). More particularly, one object of the present invention is to produce synthetic fuels from carbon dioxide (CO₂) and hydrogen (H₂). The capture of carbon dioxide and conversion thereof to a fuel base according to the invention comprises two successive steps: the conversion of the carbon dioxide and hydrogen to syngas composed mainly of CO+H₂, followed by the conversion of the syngas to synthetic hydrocarbons by the Fischer-Tropsch (FT) process. The properties of the products from the Fischer-Tropsch process can be adjusted by suitable post-treatment operations to obtain the desired fuel specifications.

### PRIOR ART

The use of the reverse water gas shift (RWGS) conversion process to convert a mixture of carbon dioxide and hydrogen to syngas CO+H₂ has been known to those skilled in the art for a very long time. The same is true for the Fischer-Tropsch synthesis process, which makes it possible to convert the syngas into a mixture of paraffins and/or olefins depending on the catalyst and operating conditions. Where paraffins are produced, it is preferable to improve certain properties thereof in order to enable them to be used for transport applications.

Sequences of unit operations have been the subject of patent applications, these sequences of unit operations aim to convert carbon dioxide into a base for fuels, often known as e-fuels.

Mention may be made, for example, of patent application US2010/0280135 A1 which describes a renewable Fischer-Tropsch synthesis process which makes it possible to produce hydrocarbons and alcohols from wind energy, residual carbon dioxide and water. The process comprises the following unit operations: electrolysis of water to produce hydrogen and oxygen, an RWGS reactor for the production of syngas, a Fischer-Tropsch synthesis in a high-temperature multitubular reactor. Various recycling options are described (e.g. recycling after separation of unconverted carbon dioxide from the RWGS, recycling the ex-FT carbon dioxide to RWGS, recycling ex-FT unconverted H₂ and CO to FT).

However, patent application US2010/0280135 A1 does not mention the possibility of advantageously integrating unit operations with a carbon dioxide capture process. There is also no mention of thermal integration between the various sources of heat energy generated by the unit operations.

Patent application US2007/0244208 A1 relates to a process for the production of high octane fuel from carbon dioxide and water. The raw material is industrial carbon dioxide and water. The final product may be high octane gasoline, high cetane diesel or other liquid hydrocarbon mixtures suitable for driving conventional combustion engines or hydrocarbons suitable for further industrial processing or commercial use. Products, such as dimethyl ether or methanol may also be withdrawn from the production line. The heat generated by exothermic reactions in the process is fully utilized, as is the heat produced by the production process and the heat produced by the reprocessing of hydrocarbons not suitable for liquid fuel.

However, patent application US2007/0244208 A1 does not mention the possibility of advantageously integrating unit operations with a carbon dioxide capture process. There is also no mention of recycling from the Fischer-Tropsch reactor in order to maximize fuel production.

Patent application US2012/0079767 A1 describes a process and system for producing syngas by combining hydrogen and carbon monoxide from separate sources while controlling the molar ratio (H₂/CO) of the syngas produced. Hydrogen is produced by electrolysis of water. Carbon monoxide is produced by reacting carbon dioxide captured in the exhaust gases of stationary combustion engines with hydrogen in an RWGS reactor. Hydrocarbon fuels are produced from syngas by Fischer-Tropsch synthesis.

However, patent application US2012/0079767 A1 does not mention the possibility of advantageously integrating unit operations with a carbon dioxide capture process. There is also no mention of recycling from the Fischer-Tropsch reactor in order to maximize fuel production.

Patent application US2007/0142481 A1 describes a process for synthesizing hydrocarbons comprising the introduction of hydrogen and carbon monoxide into a first Fischer-Tropsch reaction stage allowing the hydrogen and carbon monoxide partially to react catalytically to form hydrocarbons. At least a portion of a tail gas which comprises unreacted hydrogen and carbon monoxide, obtained from the first reaction step, is introduced into a second Fischer-Tropsch reaction step which is a two-phase high temperature catalytic Fischer-Tropsch reaction step. The hydrogen and carbon monoxide can at least partially react catalytically in the second reaction step to form gaseous hydrocarbons. This patent application is characterized by the presence of two Fischer-Tropsch reactors in series, the second one treats the unconverted syngas from the first one. There is no recycling of carbon dioxide or recycling of water.

Thus, the analysis of the prior art shows that the sequence of RWGS and Fischer-Tropsch unit operations of makes it possible to produce synthetic bases for fuels from carbon dioxide and hydrogen, it being possible in some cases for the hydrogen to be produced by electrolysis of water with a source of electricity such as solar power or wind power.

However, these documents do not provide any elements relating to the possibility of integrating, with an unexpected positive result, unit operations with the carbon dioxide capture process that provides the raw material containing the carbon source in order to produce the fuels.

### SUMMARY OF THE INVENTION

In the context described above, a first object of the present description is to overcome the problems of the prior art and to capture and upgrade carbon dioxide in the form of synthetic fuels that can be used for means of transport.

The invention relates to the capture and conversion of carbon dioxide, in order to produce a CO+H₂ syngas, and to the conversion of said syngas to synthetic hydrocarbons by the Fischer-Tropsch reaction. The characteristics of the effluents from the Fischer-Tropsch synthesis can then be adjusted by a post-treatment process (upgrading) so that they are compatible with the use for land, aviation and marine fuels. The gases produced at the outlet of the Fischer-Tropsch reactor can also be upgraded to synthetic methane (e-methane), synthetic natural gas SNG (e-SNG) or LPG (e-LPG).

Specifically, the present invention relates to a device and a process for producing synthetic fuels from carbon dioxide and hydrogen, allowing improved production of products of interest. Advantageously, the process also makes it possible, by an original thermal integration, to minimize the energy requirements for the production of said fuels.

The invention is based on the presence of a unit for separating the carbon dioxide contained in the gaseous effluent from the Fischer-Tropsch reaction section; the carbon dioxide separated from this gaseous effluent is recycled to the inlet of the RWGS reaction section. Thus, the gaseous effluent that can be sent to an air combustion section essentially no longer contains carbon dioxide.

Preferably, the presence of an air combustion unit makes it possible to produce a gaseous effluent comprising carbon dioxide, nitrogen and steam, by combustion of the gaseous hydrocarbon by-products of the process. Advantageously, the gaseous effluent can be returned to the inlet of the carbon dioxide capture unit, so as to capture the carbon dioxide and then upgrade it in the RWGS reaction unit. Advantageously, the release of heat generated by the air combustion makes it possible to supply heat energy to other units of the process, such as the RWGS reaction unit and/or the carbon dioxide capture unit, thereby limiting the external supply of heat energy required.

The energy integration of the process also makes it possible to produce electricity from heat recovery. This heat converted into electricity makes it possible to supply energy both for the electrolysis of water and for the RWGS reactor and/or the capture unit that converts carbon dioxide and hydrogen into syngas.

Advantageously, hydrogen produced by electrolysis of water can be used for the conversion of the carbon dioxide, the Fischer-Tropsch synthesis and the post-treatment. Preferably, the hydrogen needed in the process is entirely provided by a water electrolysis unit. Thus, the process according to the invention does not require an external hydrogen supply, for example produced by steam reforming of natural gas. The electrolyzer will preferably run on low-carbon electricity, which will contribute to the renewable nature of the fuels and gases produced. In addition, the water used for the production of hydrogen can originate, at least in part, from the recycling of the water produced in the various steps of the process, which has the advantage of limiting the external supply of water.

According to a first aspect, the abovementioned objects, and also other advantages, are obtained by a device for capturing and converting a feedstock containing carbon dioxide, comprising the following units:
a unit for capturing carbon dioxide from the feedstock using, for example, at least one amine-based solvent, at least one physical solvent, for instance based on polyethylene glycol dimethyl ether, and/or physical adsorption equipment operated by temperature swing adsorption, and that is suitable for producing a carbon dioxide-rich effluent;
a water electrolysis unit suitable for converting water in order to produce oxygen and hydrogen;
a reverse water gas shift RWGS reaction unit suitable for treating the carbon dioxide-rich effluent with hydrogen and producing an RWGS gas enriched in carbon monoxide and water;
a Fischer-Tropsch reaction unit suitable for: converting the RWGS gas and producing an FT effluent, and optionally generating first water vapor, generated for example by vaporizing water in an exchanger located inside the Fischer-Tropsch reaction unit, to supply thermal energy to the carbon dioxide capture unit;
a first separation unit suitable for at least partly treating the FT effluent and producing: a hydrocarbon effluent, a first water effluent which is optionally recycled at least in part to the inlet of the water electrolysis unit, and a first gaseous effluent;
a second separation unit suitable for treating the first gaseous effluent and producing: a carbon dioxide-rich gaseous effluent sent at least partially to the RWGS reaction unit and a carbon dioxide-depleted gaseous effluent optionally recycled at least partially to the Fischer-Tropsch reaction unit; and
a hydrogen reaction unit (hydrotreating and/or hydrocracking and/or hydroisomerization unit) suitable for treating the hydrocarbon effluent and producing at least one hydrocarbon cut, for example to the specifications for transport applications.

According to one or more embodiments, the device comprises an air combustion reaction unit suitable for at least partially oxidizing the carbon dioxide-depleted gaseous effluent, producing a combustion effluent comprising carbon dioxide and water, and sending the combustion effluent to the carbon dioxide capture unit.

According to one or more embodiments, the air combustion reaction unit is suitable for producing heat used to supply heat energy to the RWGS reaction unit and/or the carbon dioxide capture unit (via a feed line), for example by heat exchange in order to heat the carbon dioxide-rich effluent and/or the carbon dioxide-rich gaseous effluent and/or hydrogen, or by integrating the reaction section of the RWGS reaction unit into the chamber of the air combustion unit.

According to one or more embodiments, a portion of the hydrogen is supplied downstream of the RWGS reaction unit and upstream of the Fischer-Tropsch reaction unit.

According to one or more embodiments, a feedstock/effluent heat exchange enables the heat available in the RWGS effluent to be used to preheat the gases entering the RWGS reaction unit (gases rich inCO₂ and H₂).

According to one or more embodiments, the device comprises a first heat exchanger suitable for generating a second steam by heat exchange between water and the RWGS gas which can be used for example to supply thermal energy to the carbon dioxide capture unit. According to one or more embodiments, the device comprises a first turbine for treating at least one portion of the carbon dioxide-depleted gaseous effluent separated by the first separation unit in order to generate electricity.

According to one or more embodiments, a second turbine is suitable for treating at least in part, the first steam and/or the second steam in order to produce electricity.

According to one or more embodiments, the electricity is used to supply heat energy to the RWGS reaction unit and/or the carbon dioxide capture unit and/or the water electrolysis unit.

According to one or more embodiments, the electricity is used to supply heat energy to the regeneration section of the carbon dioxide capture unit.

According to one or more embodiments, the water electrolysis unit treats water from a makeup line and/or from the RWGS gas and/or from the FT effluent.

According to one or more embodiments, the water from the RWGS gas is at least partly or completely separated by a third separation unit in order to be sent to the water electrolysis unit. According to one or more embodiments, the carbon dioxide-rich effluent and/or the carbon dioxide-rich gaseous effluent are purified, separately or after mixing, before being introduced into the RWGS reaction unit. In one or more embodiments, the RWGS gas is purified before being introduced into the Fischer-Tropsch reaction unit, upstream or downstream of the third separation unit. According to one or more embodiments, the first water effluent is purified before being introduced into the water electrolysis unit. The effluent purification steps aim to at least partially eliminate the sulfur-containing and nitrogen-containing compounds, halogens, heavy metals and transition metals. The main technologies for the purifying the gases are: adsorption, absorption, catalytic reactions.

According to one or more embodiments, the device comprises a carbon dioxide separation unit positioned between the RWGS reaction unit and the Fischer-Tropsch reaction unit. Advantageously, the size of the FT reaction unit 13 can thus be reduced.

According to a second aspect, the abovementioned objects, and also other advantages, are obtained by a process for capturing and converting carbon dioxide, comprising the following steps:
treating the feedstock in a carbon dioxide capture unit in order to produce a carbon dioxide-rich effluent;
converting water in a water electrolysis unit in order to produce oxygen and hydrogen;
treating the carbon dioxide-rich effluent with hydrogen in a reverse water gas shift RWGS reaction unit, in order to produce an RWGS gas enriched in CO and in water;
converting the RWGS gas in a Fischer-Tropsch reaction unit in order to produce an FT effluent;
optionally generating a first steam in the Fischer-Tropsch reaction unit in order to supply thermal energy to the carbon dioxide capture unit;
treating the FT effluent in a first separation unit in order to produce at least one hydrocarbon effluent, a first water effluent and a first gaseous effluent;
treating the first gaseous effluent in a second separation unit in order to produce a carbon dioxide-depleted gaseous effluent and a carbon dioxide-rich gaseous effluent which is recycled to the inlet of the RWGS section;
optionally oxidizing at least one portion of the carbon dioxide-depleted gaseous effluent, for example after expansion in a turbine, in an air combustion reaction unit in order to produce a combustion effluent comprising carbon dioxide and water;
optionally sending the combustion effluent to the carbon dioxide capture unit; and
treating the hydrocarbon effluent in a hydrogen reaction unit in order to produce at least one hydrocarbon cut, for example to the specifications required for transport applications.

According to one or more embodiments, the RWGS reaction unit comprises at least one reactor used under at least one of the following operating conditions:
temperature of between 700° C. and 1200° C., preferentially between 800° C. and 1100° C. and even more preferentially between 850° C. and 1050° C.;
pressure of between 0.1 MPa and 10 MPa, preferentially between 0.1 MPa and 5 MPa and more preferentially between 0.1 MPa and 3.5 MPa;
space velocity of the gas at the inlet of the reactor of between 5000 NL/kg_{cata}/h and 40 000 NL/kg_{cata}/h;
catalyst comprising a metal or a combination of metals selected from the group consisting of the elements Ni, Cu, Fe, Co, Pt, Pd, Ru, Ag and Au. According to one or more embodiments, the catalyst for the RWGS reaction comprises a support, for example based on alumina, silica, silica-alumina, siliceous alumina.

According to one or more embodiments, the FT reaction unit comprises at least one reactor used under at least one of the following operating conditions:
temperature of between 170° C. and 280° C., preferably between 190° C. and 260° C. and preferentially between 210° C. and 240° C.;
absolute pressure of between 0.1 MPa and 6.0 MPa and preferably between 1.5 MPa and 3.5 MPa and preferentially between 2.0 MPa and 3.0 MPa;
catalyst comprising cobalt or iron, preferably cobalt, the catalyst optionally comprising a support, for example based on alumina, silica, silica-alumina, siliceous alumina or titanium. According to one or more embodiments, the air combustion reaction unit comprises at least one reactor used under at least one of the following operating conditions:
absolute pressure of between 0.1 MPa and 4 MPa;
temperature of between 600° C. and 2000° C., preferably between 800° C. and 1800° C., and preferentially between 900° C. and 1500° C.;
presence of air used for the combustion, with an aeration rate of between 1 and 2, and preferably of at least 1.2, the aeration rate being defined as the ratio of the molar flow rate of air injected to the theoretical air flow rate for complete oxidation of all the fuels.

According to one or more embodiments, the second separation unit is a unit for separating carbon dioxide by means of a membrane and/or by absorption in a solvent and/or by adsorption on a solid.

Embodiments of the device and process according to the abovementioned aspects, and also other features and advantages, will become apparent on reading the description which follows, which is given purely by way of non-limiting illustration, and with reference to the following drawing.

### LIST OF THE FIGURES

FIG. 1 shows a schematic representation of a device according to the present invention comprising a second separation unit for producing a carbon dioxide-rich gaseous effluent sent to the RWGS reaction unit.

### DESCRIPTION OF THE EMBODIMENTS

Embodiments of the device according to the first aspect and of the process according to the second aspect will now be described in detail. In the following detailed description, numerous specific details are disclosed in order to provide a deeper understanding of the device. However, it will be apparent to those skilled in the art that the device can be used without these specific details. In other cases, well-known features have not been described in detail in order to avoid unnecessarily complicating the description.

In the present description, the term “to comprise” is synonymous with (means the same as) “to include” and “to contain”, and is inclusive or open and does not exclude other elements that are not stated. It is understood that the term “to comprise” includes the exclusive and closed term “to consist of”. Moreover, in the present description, an effluent comprising essentially or solely a compound A corresponds to an effluent comprising at least 95% by weight, preferably at least 98% by weight, very preferably at least 99% by weight, of compound A.

In the present description, the term “physical solvent” is synonymous with (means the same thing as) solvent that forms a weak bond (e.g. hydrogen bond, van der Waals bond) with the solute or solvent that does not form a strong bond (e.g. covalent bond, ionic bond) with the solute.

The present invention can be defined as a device and a process comprising a sequence of unit operations for producing synthetic hydrocarbons, such as synthetic fuels, for example gasoline, kerosene, gas oil and/or naphtha or lubricant bases, preferably of very high quality, from carbon dioxide from a capture unit.

The device and the process according to the invention are notably characterized in that they comprise and use units for carbon dioxide capture, for reverse water gas shift (RWGS) conversion, for Fischer-Tropsch (FT) synthesis, and for hydrogen treatment (hydrotreating, and/or hydrocracking and/or hydroisomerization) of the hydrocarbon cuts from the FT reaction unit, for separating the carbon dioxide from the gaseous effluents obtained from the Fischer-Tropsch process, and optionally for combustion of the gaseous hydrocarbon by-products of the process (RWGS and Fischer-Tropsch synthesis and post-treatment) after the carbon dioxide has been separated from them. Advantageously, the hydrogen required can be produced by a water electrolysis unit, it being possible for said water to originate from the RWGS and Fischer-Tropsch reaction units.

One of the characteristics of the present invention can be summarized by the use of carbon dioxide for the production of synthetic fuels, gasoline, kerosene, gas oil and/or naphtha or lubricant bases of very high quality. The present invention is also based on the presence of a carbon dioxide separation unit to extract and recycle the carbon dioxide from the other gaseous hydrocarbon by-products of the process.

An air combustion unit may advantageously be provided for treating the carbon dioxide-depleted gaseous hydrocarbon by-products in order to produce a carbon dioxide-rich gaseous effluent so as to improve the production of products of interest.

In addition, the release of heat generated by the combustion can advantageously be used to provide heat energy to the RWGS reaction unit (8) and/or the carbon dioxide capture unit (2). This input of heat energy can be carried out for example by heat exchange with:
the combustion gas within the combustion chamber; and/or
the high-temperature gaseous effluent downstream of the combustion chamber; and/or
steam generated by the air combustion reaction unit and/or by heat exchange with the gaseous combustion effluent.

According to one or more embodiments, the present invention also makes it possible to minimize the amount of carbon energy external to the process and thus the impact on the environment, by means of an original energy integration based on the use of the heat at the outlet of the Fischer-Tropsch reaction unit and optionally the RWGS reaction unit, to desorb carbon dioxide, for example complexed with amine in the carbon dioxide capture unit and more particularly in a solvent regeneration unit.

According to one or more embodiments, electricity can also be produced by a turbine fed by an effluent from the Fischer-Tropsch reaction unit and/or by steam (e.g. produced at the outlet of the Fischer-Tropsch reaction unit and/or the RWGS reaction unit), this electricity making it possible, for example, to supply heat energy to the device according to the invention, for example to the RWGS reaction unit.

Thus, the combination of units for the capture and chemical conversion of carbon dioxide, with preferably an original thermal integration, makes it possible to produce bases for fuels, and in particular for fuel for the aviation sector, while minimizing the environmental impact of the process.

Preferably, the use of the water electrolysis unit to treat the water produced by the RWGS reaction unit and/or the Fischer-Tropsch unit also makes it possible to minimize the environmental impact of the process.

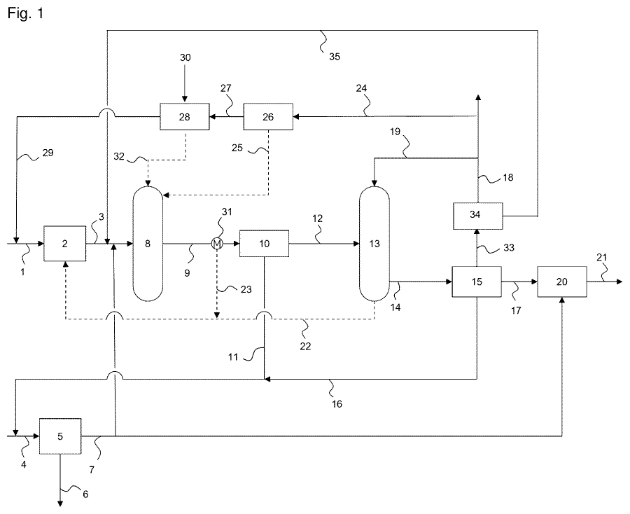

With reference to FIG. 1, the device for converting carbon dioxide to liquid hydrocarbons comprises:
a carbon dioxide capture unit 2 suitable for treating a feedstock 1 containing carbon dioxide and producing a carbon dioxide-rich (gaseous) effluent 3 (i.e. which is enriched in carbon dioxide compared to the feedstock 1);
a water electrolysis unit 5 suitable for treating water 4 (fresh or recycled) in order to produce oxygen 6 and hydrogen 7;
an RWGS reaction unit 8 suitable for at least partially converting the carbon dioxide from the carbon dioxide-rich effluent 3 into a CO-rich RWGS gas 9 (i.e., syngas which is enriched in CO (and water) compared to the carbon dioxide-rich effluent 3);
a Fischer-Tropsch (FT) reaction unit 13 suitable for converting the RWGS gas 9 and producing a Fischer-Tropsch (FT) effluent 14, and optionally suitable for generating a first steam 22, generated for example by vaporizing water in an exchanger located inside the FT reaction unit 13, in order to supply thermal energy to the carbon dioxide capture unit 2;
a first separation unit 15 suitable for at least partly treating the FT effluent 14 and producing: at least a hydrocarbon effluent 17, a first gaseous effluent 33 (off-gas), and a first water effluent 16, product of the Fischer-Tropsch synthesis obtained from the condensation of gaseous water under the operating conditions of the Fischer-Tropsch reaction;
a second separation unit 34 for producing a carbon dioxide-depleted gaseous effluent 18 and a carbon dioxide-rich gaseous effluent 35 from the first gaseous effluent 33, and recycling the carbon dioxide-rich gaseous effluent 35 to the inlet of the RWGS section 8;
optionally an air combustion reaction unit 28 suitable for oxidizing at least a portion 24 of the carbon dioxide-depleted gaseous effluent 18 separated by the second separation unit 34, producing a combustion effluent 29 comprising carbon dioxide and water, and sending the combustion effluent 29 to the carbon dioxide capture unit 2;
a hydrogen reaction unit 20 (hydrotreating and/or hydrocracking and/or hydroisomerization unit) suitable for treating the hydrocarbon effluent 17 with hydrogen 7 and separating at least one hydrocarbon cut 21, comprising for example at least one of the following cuts: naphtha, gasoline, kerosene, gas oil, and lubricant base;
optionally at least one heat exchanger 31 suitable for generating a second steam 23 by heat exchange between water and the RWGS gas 9, which can be used for example to supply thermal energy to the carbon dioxide capture unit 2; and
preferably a separation unit 10 suitable for treating the RWGS gas 9 in order to produce an RWGS gas 12 that is depleted in water (compared to the RWGS gas 9), and that is sent to the FT reaction unit 13 instead of the RWGS gas 9, and sending a second water effluent 11 to the water electrolysis unit 5.

Advantageously, the FT reaction unit 13 and optionally the first heat exchanger 31 are suitable for producing steam by heat exchange. Advantageously, the use of steam makes it possible to supply energy to the carbon dioxide capture unit 2, for example by regenerating an amine-based solvent (or a physical solvent) loaded with carbon dioxide in a regeneration unit of the carbon dioxide capture unit 2, or by feeding temperature swing adsorption equipment.

In order to avoid unnecessarily complicating the description and the figures, it will be apparent to those skilled in the art that the water supplies to the FT reaction unit 13 and to the heat exchanger 31 for generating steam have not been described in detail. The same is true for the water outlet from the carbon dioxide capture unit 2.

### Carbon Dioxide Capture Unit

The carbon dioxide capture unit 2 makes it possible to separate the carbon dioxide from the rest of the feedstock 1. Such a carbon dioxide capture unit conventionally makes it possible to supply CO₂ which can be compressed for upgrading or for storage. According to one or more embodiments, the feedstock 1 comprises at least 0.04 vol % of carbon dioxide, preferably at least 2 vol % of carbon dioxide, very preferably at least 10 vol % of carbon dioxide.

According to one or more embodiments, the feedstock 1 comprises or consists of combustion flue gases. According to one or more embodiments, the feedstock 1 comprises gaseous effluents from at least one unit chosen from the group consisting of: a refinery, an incinerator, a petrochemical unit, a chemical unit, a thermal power plant, a paper mill, an ethanol plant and a sugar refinery. According to one or more embodiments, the feedstock 1 comprises gaseous effluents from a cement plant, and/or gaseous effluents from a lime production unit, and/or gaseous effluents from blast furnaces. According to one or more embodiments, the combustion fumes originate from a combustion chamber (e.g. a boiler) designed for burning a fuel, such as coal, natural gas, fuel oil, biogas, biomass, organic waste, municipal waste, with an oxidizer, usually air.

According to one or more embodiments, the feedstock 1 comprises or consists of biogas, natural gas, syngas, refinery gas, biomass fermentation gas, cement plant gas and/or blast furnace gas.

The carbon dioxide can also be the carbon dioxide present in the air. According to one or more embodiments, the feedstock 1 comprises or consists of air. For example, the carbon dioxide capture unit 2 may include a direct air capture (DAC) device.

For the capture, several agents can be used, such as solvents and solids. According to the invention, the carbon dioxide capture unit 2 uses at least one amine-based solvent, and/or at least one physical solvent, for instance based on polyethylene glycol dimethyl ether, and/or temperature swing adsorption (physical adsorption) equipment.

One widespread carbon dioxide capture technology is based on the phenomenon of absorption, i.e. the transition of a chemical species from a gas to a liquid. The gas containing the impurities, or the species to be separated, is sent to a column where it is brought into contact with a liquid solvent, it being possible for the two streams to be used in various hydrodynamic configurations (co-current, cross-current or counter-current, the latter solution being preferred for reasons of favorable thermodynamic equilibrium). This absorption is carried out using an absorbent solution, including a chemical solvent or a physical solvent, this distinction being linked to whether or not there is a chemical reaction between the component absorbed and the solvent.

Physical absorption is preferred with a view to minimizing the energy cost of the process; this is particularly suitable in the case of high partial pressure of the species to be separated.

Chemical absorption is preferred in the case of high dilution and low partial pressure of the species to be separated and/or in the case where a high recovery rate of this species is desired, or finally if achieving a strict specification regarding the maximum allowable concentration of this species in the gas stream once washed is desired. Thus, for the capture of carbon dioxide from (industrial) flue gases with a low carbon dioxide concentration value, typically between 3% and 15% by volume (typically in gases at low pressure), washing by chemical absorption, for example using an amine solvent, for example of alkanolamine type, is well suited.

The absorbent solutions commonly used today are aqueous solutions containing one or more reactive compounds or having a physicochemical affinity with the acid compounds. The reactive compounds can for example, and nonlimitingly, be amines (primary, secondary, tertiary, cyclic or noncyclic, aromatic or nonaromatic, saturated or unsaturated), alkanolamines, polyamines, amino acids, alkali metal salts of amino acids, amides, ureas, phosphates, carbonates or borates of alkali metals. According to one or more embodiments, the absorbent solution is an aqueous solution comprising one or more reactive compounds with an amine function and the structure of which is described from page 6, line 1 to page 7, line 3, of patent application WO2007/104856.

According to one or more embodiments, the reactive compounds represent from 10% by weight to 90% by weight, preferably between 20% by weight and 50% by weight, very preferably between 25% by weight and 40% by weight, of the total weight of the absorbent solution.

Chemical absorption in amine solvents is based on acid-base equilibria, low temperature favoring the reaction between the basic amine and acidic carbon dioxide, high temperature favoring the reverse reaction. Thus, amine processes, using for example an aqueous phase containing 20-50% by weight of amine(s), can use two columns (not shown) in which the solvent circulates from one to the other. In the first column, called the absorber, the stream to be washed (i.e. the feedstock 1) is brought into contact with the amine solvent at low temperature. The amine solvent flows into the column and captures the carbon dioxide. At the bottom of the column, the amine solvent (“rich” solvent) reaches a predetermined loading rate, the ratio between the number of moles of carbon dioxide captured and the number of moles of amines; at the top of the column, the gaseous stream exits, itself at predetermined specifications, namely a carbon dioxide content for example close to 10 times lower than the initial content in the flue gases. The rich solvent is sent to the second column, called the regenerator, the operation of which is similar to that of a distillation column, operating at high temperature. The regenerated amine solvent (“lean” solvent) can itself be sent back to the absorber. The amine solvent thus circulates continuously in a closed loop from one column to the other, preferably passing through a feedstock/effluent heat exchanger to cool the lean solvent and preheat the rich solvent while saving energy throughout the process.

According to one or more embodiments, the regenerator operates at a high temperature of between 90° C. and 250° C., preferably between 110° C. and 240° C., very preferably between 120° C. and 200° C. at the bottom of the column.

The carbon dioxide released from the regenerator can then optionally be compressed and upgraded. According to one or more embodiments, the carbon dioxide-rich effluent 3 comprises at least 90 vol % of carbon dioxide, preferably at least 95 vol % of carbon dioxide, very preferably at least 98 vol % of carbon dioxide. According to one or more embodiments, the carbon dioxide-rich effluent 3 has a temperature of between 20° C. and 250° C., preferably between 30° C. and 200° C., very preferably between 40° C. and 150° C., on leaving the carbon dioxide capture unit 2. According to one or more embodiments, the carbon dioxide-rich effluent 3 has a pressure of between 0.20 MPa and 4 MPa, preferably between 0.30 MPa and 3.5 MPa, very preferably between 0.4 MPa and 3 MPa, on leaving the carbon dioxide capture unit 2.

An essential aspect of the operations for solvent treatment of industrial flue gases is the step of regenerating the separating agent. Depending on the type of absorption (physical and/or chemical), regeneration by expansion, and/or by distillation and/or by entrainment by a vaporized gas, known as “stripping gas”, is generally envisaged.

One of the main limitations of the solvents commonly used today is the need to use high flow rates of absorbent solution, which leads to a high energy consumption for solvent regeneration, and also large sizes of the equipment (columns, pumps, etc.). This is particularly true in the case where the partial pressure of carbon dioxide is low. Such an energy consumption represents a considerable operating cost for the carbon dioxide capture process. The regeneration energy depends on the nature of the amines and on the partial pressure of carbon dioxide and is typically between 2 GJ/t and 4 GJ/t of carbon dioxide captured. New capture processes tend to reduce this energy in order to tend toward values below 2 GJ/t of carbon dioxide. In the context of air treatment, since the concentrations of carbon dioxide are very low, the energies consumed are very high, of the order of 5 GJ/t of carbon dioxide to 7.5 GJ/t of carbon dioxide.

Another possible implementation is based on the principle of adsorption by means of a solid adsorbent having a strong chemical affinity for carbon dioxide. To ensure a continuous mode of operation, the processes operate with several reactors in parallel. The carbon dioxide is adsorbed on the solid adsorbent and the stream to be treated (i.e., the feedstock 1) becomes depleted as it advances through the bed of solid, and, at the outlet, the stream no longer or scarcely contains any carbon dioxide. However, the solid adsorbent is gradually saturated and can no longer adsorb carbon dioxide. The stream to be treated is then sent to another reactor containing a solid adsorbent that is not saturated with carbon dioxide, and the capture operation continues. At the same time, the reactors saturated with carbon dioxide undergo a regeneration operation:
a rise in temperature refers to temperature swing adsorption (TSA); and
a partial vacuum then refers to pressure swing adsorption (or VPSA for vacuum pressure swing adsorption, or simply VSA or PSA, optionally in the presence of a gas that promotes desorption).

The barrier to TSA processes is the large amount of heat required for the regeneration. The thermal integration proposed in the present invention makes it possible to remove this barrier.

According to one or more embodiments, the solid adsorbent for carbon dioxide capture is selected from the following compounds: activated carbon, zeolites, aluminas, silicas, synthetic fibers with or without impregnated amines, metal-organic framework (MOF) solids, and supported alkali metal carbonates. These solid adsorbents are increasingly used for the capture of carbon dioxide from air. The energy for regeneration of the adsorbents with physisorption in these cases, for example on zeolites, is of the order of 0.6 to 0.9 GJ/t of carbon dioxide. For solid supported amines, the regeneration energy is between 5.4 and 7.2 GJ/t of carbon dioxide.

Advantageously, the energy needed for the regeneration of the amine solvent and/or the temperature rise of the solid adsorbent can be supplied at least partially by the first steam 22 and optionally the second steam 23. This input of energy to the carbon dioxide capture unit 2 makes it possible to maximize the energy efficiency of the process.

According to one or more embodiments, the temperature of the steam 22 and/or 23 is at least 110° C., preferably at least 120° C., very preferably at least 130° C., for example at the outlet of the heat exchanger 31 and/or the FT reaction unit 13. According to one or more embodiments, the temperature of the steam 22 and/or 23 is between 110° C. and 270° C., preferably between 120° C. and 260° C., very preferably between 130° C. and 220° C., for example at the outlet of the heat exchanger 31 and/or the FT reaction unit 13. According to one or more embodiments, the steam 22 and/or 23 has a pressure of between 0.1 MPa and 4 MPa, preferably between 0.1 MPa and 3.5 MPa, very preferably between 0.1 MPa and 1.7 MPa, for example at the outlet of the heat exchanger 31 and/or the FT reaction unit 13.

### Water Electrolysis Unit

The water electrolysis unit 5 treats water 4 from: a makeup line and/or from the optional third separation unit 10 and/or from the first separation unit 15.

According to one or more embodiments, the water electrolysis unit 5 comprises a pre-treatment section suitable for extracting oxygen-containing compounds from the water 4, for example from the first water effluent 16.

According to one or more embodiments, the water electrolysis unit 5 comprises at least one alkaline electrolyser. Other electrolyser technologies can be used for the water electrolysis unit, such as proton exchange membrane (PEM) electrolysis, solid oxide electrolysis (SOE), or anion exchange membrane (AEM) electrolysis. The operating conditions (temperature, pressure, nature of the electrolyte, electrodes and diaphragm/membrane) are then specific to each technology.

According to one or more embodiments, the water electrolysis unit 5 comprises at least one reactor used under at least one of the following operating conditions:

### Alkaline Electrolyser:

temperature of between 60° C. and 90° C.,
pressure of between 0.1 MPa and 20 MPa, preferably between 0.1 MPa and 4 MPa,
electrolyte comprising KOH,
electrodes comprising a metal alloy,
diaphragm comprising asbestos, polytetrafluoroethylene and/or nickel oxide;
Proton exchange membrane (PEM) electrolyser:
temperature of between 50° C. and 80° C.,
pressure of between 0.1 MPa and 20 MPa, preferably between 1.8 MPa and 5.5 MPa,
electrolyte comprising a polymer membrane,
electrodes comprising a metal alloy;
Solid oxide electrolyser (SOE):
temperature of between 800° C. and 900° C.,
pressure of between 0.1 MPa and 2 MPa, preferably between 0.1 MPa and 0.5 MPa,
electrolyte comprising a ceramic (e.g. perovskite) membrane,
electrodes comprising a metal alloy;
Anion exchange membrane (AEM) electrolyser:
temperature of between 50° C. and 70° C.,
pressure of between 0.1 MPa and 20 MPa, preferably between 0.1 MPa and 3.5 MPa,
electrolyte comprising a polymer membrane,
electrodes comprising a metal alloy.

According to one or more embodiments, the oxygen 6 produced by the water electrolysis unit 5 comprises between 99.0% by weight and 99.8% by weight of O₂ (after drying).

According to one or more embodiments, the hydrogen 7 produced by the water electrolysis unit 5 comprises between 99.5% by weight and 99.999% by weight of H₂ (after drying).

According to one or more embodiments, the water electrolysis unit 5 is based on a solid oxide electrolyser (SOE) technology for which at least a portion of the water 4 can be in the form of steam supplied at least partially by the first steam 22 and optionally the second steam 23. This input of energy to the electrolysis unit 5 makes it possible to improve the energy efficiency of the process.

### RWGS Reaction Unit

The RWGS reaction unit 8 produces an RWGS gas 9 (syngas) which is enriched in CO (and depleted in hydrogen) compared to the group of carbon dioxide-rich effluents 3 and 35 that contain unconverted carbon dioxide and water. The hydrogen 7 required for the RWGS reaction comes from the water electrolysis unit 5.

According to one or more embodiments, the RWGS reaction unit 8 comprises at least one reactor used under at least one of the following operating conditions:
temperature of between 700° C. and 1200° C., preferentially between 800° C. and 1100° C. and even more preferentially between 850° C. and 1050° C.;
pressure of between 0.1 MPa and 10 MPa, preferentially between 0.1 MPa and 5 MPa and more preferentially between 0.1 MPa and 3.5 MPa;
space velocity of the gas at the inlet of the reactor of between 5000 NL/kg_{cata}/h and 40 000 NL/kg_{cata}/h;
catalyst based on elements Ni, Cu, Fe, Co or precious metals such as Pt, Pd, Ru, Ag and Au. According to one or more embodiments, the catalyst for the RWGS reaction comprises a support, for example based on alumina, silica, silica-alumina or siliceous alumina.

According to one or more embodiments, the amount of hydrogen at the inlet of the RWGS reaction unit 8 is adjusted so that the H₂/CO molar ratio at the outlet of the RWGS reaction unit 8 is compatible with the requirement of the FT unit, i.e. between 0.5 and 4, preferably between 1 and 3, more preferably between 1.5 and 2.5.

According to one or more embodiments, the RWGS gas 9 has a temperature at the outlet of the RWGS reaction unit 8 of at least 700° C., preferably at least 750° C., very preferably at least 800° C.

In one or more embodiments, at least one portion of the RWGS gas 9 supplies energy to the regeneration unit of the carbon dioxide capture unit 2, by means of the first heat exchanger 31 producing the second steam 23 by (indirect) heat exchange between water (not shown) and the RWGS gas 9, preferably directly at the outlet of the RWGS reaction unit 8.

The RWGS gas 9 is preferably sent to the third separation unit 10.

### Fischer-Tropsch Reaction Unit

According to the invention, in the FT reaction unit 13, the carbon monoxide and hydrogen present in the RWGS gas 9 (preferably depleted in water) react to produce a stream comprising an FT effluent 14 comprising unconverted syngas, carbon dioxide, gaseous and liquid hydrocarbon products and water.

According to one or more embodiments, the RWGS gas 9 (preferably depleted in water) sent to the FT reaction unit 13 comprises carbon monoxide and hydrogen with an H₂/CO molar ratio of between 0.5 and 4, preferably between 1 and 3, more preferably between 1.5 and 2.5.

According to one or more embodiments, the amount of hydrogen upstream (e.g. at the inlet) of the FT reaction unit 13 is adjusted, for example by means of an optional hydrogen supply, so that the H₂/CO molar ratio is as defined above.

The FT reaction unit 13 is used in a reaction unit comprising one or more suitable reactors, the technology of which is known to those skilled in the art. This may be, for example, one or more multitubular fixed bed reactors, or one or more slurry bubble column reactors, or one or more microchannel reactors.

According to one or more embodiments, the FT reaction unit uses one or more bubble-column reactors. As the synthesis is highly exothermic, this embodiment makes it possible, inter alia, to improve the heat control of the reactor and to create only small pressure drops.

The catalyst used in this Fischer-Tropsch synthesis is generally any catalytic solid known to those skilled in the art for performing the Fischer-Tropsch synthesis. According to one or more embodiments, the catalyst used in the Fischer-Tropsch synthesis comprises cobalt or iron, preferably cobalt. The catalyst used is generally a supported catalyst. The support may be, for example, based on alumina, silica, silica-alumina, siliceous alumina or titanium.

According to one or more embodiments, the FT reaction unit 13 comprises at least one reactor used under at least one of the following operating conditions:
temperature of between 170° C. and 280° C., preferentially between 190° C. and 260° C. and preferentially between 210° C. and 240° C.,
absolute pressure of between 1.0 MPa and 6.0 Mpa, preferably between 1.5 MPa and 3.5 MPa and preferentially between 2.0 MPa and 3.0 MPa.

The FT effluent 14 is sent to the first separation unit 15. According to one or more embodiments, the FT effluent 14 has a temperature at the outlet of the FT reaction unit 13 of at least 170° C., preferably at least 190° C., very preferably at least 210° C.

According to one or more embodiments, the FT reaction unit 13 is suitable for producing the first steam 22 and supplying thermal energy to the carbon dioxide capture unit 2. The first steam 22 is generated, for example, by vaporizing water (not shown) in a heat exchanger located inside the FT reaction unit 13 making it possible to eliminate heat energy from the Fischer-Tropsch reaction, which is an exothermic reaction.

### First Separation Unit

In the first separation unit 15, at least one (first) portion of the FT effluent 14 is treated to produce:
hydrocarbon effluent 17 (depleted in water compared to the FT effluent 14),
the first gaseous effluent 33, and
the first water effluent 16.

According to one or more embodiments, a second portion of the FT effluent 14 is sent directly to the hydrogen reaction unit 20. Preferably, said second portion of the FT effluent 14 is a liquid fraction, preferably containing little or no water.

At the outlet of the first separation unit 15, the hydrocarbon effluent 17 is sent to the hydrogen reaction unit 20, and the first water effluent 16 is optionally sent to the water electrolysis unit 5 by means of a first recycling line.

According to one or more embodiments, the hydrocarbon effluent 17 comprises: n-paraffins, olefins and oxygenated compounds resulting from the condensation of the gaseous hydrocarbons under the operating conditions of the Fischer-Tropsch reaction.

According to one or more embodiments, the hydrocarbon effluent 17 comprises less than 5% by weight of water, preferably less than 2% by weight of water, very preferably less than 1% by weight of water.

According to one or more embodiments, the first water effluent 16 is produced from the Fischer-Tropsch synthesis resulting from the condensation of the gaseous water under the operating conditions of the Fischer-Tropsch reaction.

According to one or more embodiments, the first gaseous effluent 33 comprises unconverted synthesis gas, carbon dioxide and gaseous hydrocarbons such as (predominantly) C₁ to C₄ paraffins, C₂ to C₄ olefins, and C₁ to C₃ oxygenated compounds.

### Second Separation Unit

In the second separation unit 34, the first effluent 33 from the first separation unit 15 is treated in order to produce:
the carbon dioxide-depleted gaseous effluent 18; and
the gaseous effluent 35 which is rich in carbon dioxide, relative to the carbon dioxide content of the first effluent 33.

According to a first embodiment, the second separation unit 34 is a membrane separation unit. Membrane separation processes were initially not widely recommended for post-combustion carbon dioxide capture, processes of gas-liquid absorption in a chemical solvent being considered the most mature and most suitable technology for carrying out this operation. However, the latest technologies allow carbon dioxide to be separated economically with membranes (dense polymers, inorganic materials, hybrid matrices, liquid membranes). Reference may be made to the review article: Oil Gas Sci. Technol.—Rev. IFP Energies nouvelles, Volume 69, Number 6, November-December 2014. The main performance is a capture rate and a carbon dioxide purity of more than 90%.

According to a second embodiment, the second separation unit 34 is a carbon dioxide capture unit based on absorption of the carbon dioxide in a solvent.

According to a third embodiment, the second separation unit 34 is a is a carbon dioxide capture unit based on adsorption of the carbon dioxide on a solid.

According to one or more embodiments, a (first) portion 19 of the carbon dioxide-depleted gaseous effluent 18 is sent to the FT reaction unit 13 by means of a second recycle line.

According to one or more embodiments, at least a (second) portion 24 of the carbon dioxide-depleted gaseous effluent 18 is treated by the first turbine 26 to produce electricity; the gas 27 leaving the first turbine 26 is sent to the air combustion reaction unit 28.

According to one or more embodiments, a (third) portion of the carbon dioxide-depleted gaseous effluent 18 is recycled to the RWGS reaction unit 8 (not shown) in order to be converted to syngas and thereby improve the mass yield of the process line.

According to one or more embodiments, a (fourth) portion of the carbon dioxide-depleted gaseous effluent 18 is sent to an independent syngas production unit (not shown), for example of the following type:
partial oxidation (or POx);
steam methane reforming (or SMR);
autothermal reforming (or ATR);
enhanced heat transfer reforming (or EHTR).

According to one or more embodiments, said syngas produced in the independent unit is recycled to the inlet or outlet of the RWGS reaction unit 8.

### Air Combustion Reaction Unit

According to one or more embodiments, at least a portion 24 of the carbon dioxide-depleted gaseous effluent 18 is sent to the air combustion reaction unit 28, in which the hydrocarbon compounds, carbon monoxide and hydrogen present (i.e. CO, H₂, paraffins and olefins 1 to 7 carbon atoms per molecule, and alcohol compounds with 1 to 3 carbon atoms per molecule) are converted at least partially to carbon dioxide and water in the presence of air 30, to produce a combustion gas comprising (e.g. essentially) carbon dioxide and water.

According to one or more embodiments, the air combustion reaction unit 28 comprises at least one reactor used under at least one of the following operating conditions:
absolute pressure of between 0.1 MPa and 4 MPa;
temperature of between 600° C. and 2000° C., preferably between 800° C. and 1800° C., and preferentially between 900° C. and 1500° C.;
presence of air 30 with an aeration rate of between 1 and 2, and preferably of at least 1.2, in particular to limit the concentration of unburnt CO and H₂ gases.

The aeration rate is defined as the ratio of the molar flow rate of air injected to the theoretical air flow rate for complete oxidation of all the fuels.

According to one or more embodiments, a “light” hydrocarbon cut (not shown) obtained from the hydrogen reaction unit 20 is sent at least partially to the air combustion reaction unit 28 (not shown). According to one or more embodiments, the hydrocarbon cut comprises gaseous hydrocarbons such as (predominantly) C₁ to C₄ paraffins, C₂ to C₄ olefins, and C₁ to C₃ oxygen-containing compounds.

According to one or more embodiments, the combustion gas produced in the air combustion reaction unit 28 has a temperature of between 600° C. and 2000° C. and preferably between 800° C. and 1800° C. and preferentially between 900° C. and 1500° C., and an absolute pressure of between 0.1 MPa and 4 MPa.

The combustion gas produced in the air combustion reaction unit 28 being at high temperature makes it possible, via the feed line 32, to supply a portion of the heat energy needed to the RWGS reaction unit 8 and/or the carbon dioxide capture unit 2.

According to one or more embodiments, the air combustion reaction unit 28 is suitable for producing heat used to supply heat energy to the RWGS reaction unit 8 and/or the carbon dioxide capture unit 2 (via a feed line 32), for example by heat exchange in order to heat the carbon dioxide-rich effluent 3 and/or the carbon dioxide-rich gaseous effluent 35 and/or the hydrogen 7, or integrating the reaction section of the RWGS reaction unit 8 into the chamber of the air combustion unit 28.

The heat energy may be supplied, for example, by heat exchange with the steam produced by the air combustion reaction unit 28 and/or heat exchange within the combustion chamber of the air combustion reaction unit 28 and/or heat exchange with the high-temperature gaseous effluent downstream of the air combustion reaction unit 28. For example, after transfer of heat energy, for example to the RWGS reaction 8, from the combustion gas in the combustion chamber, the residual heat contained in the combustion effluent 29 at the outlet of the air combustion reaction unit 28 can be used to produce steam, sent for example to the carbon dioxide capture unit 2.

Advantageously, the air combustion reaction unit 28 makes it possible to convert substantially all of the hydrocarbon by-products of the process into carbon dioxide, and therefore to upgrade them in the form of the desired products. Thus, the yield of desired products of the process according to the invention is improved.

The combustion effluent 29 at the outlet of the air combustion reaction unit 28 is recycled to the inlet of the carbon dioxide capture unit 2.

### Hydrogen Reaction Unit

The hydrocarbon effluent 17 is sent to the hydrogen reaction unit 20 to undergo a hydrotreating and/or hydrocracking and/or hydroisomerization reaction, in which one or more hydrocarbon cuts 21 can be upgraded, in particular synthetic fuels, namely gasoline, kerosene, gas oil, and/or other hydrocarbon products, such as naphtha, or lubricant bases, of very high quality (essentially free of sulfur, aromatics, nitrogen). One possible option is the production of paraffinic cuts, base products for petrochemical processes, for example the production of a C10-C13 cut intended for the production of linear alkyl benzene (LAB), or alternatively waxes for various industrial applications.

According to one or more embodiments, the hydrogen reaction unit 20 comprises at least one reactor used under at least one of the following operating conditions:
temperature of between 250° C. and 450° C., more preferentially of between 280° C. and 450° C., and even more preferentially of between 320° C. and 420° C.;
pressure of between 0.2 MPa and 15 MPa, preferably between 0.5 MPa and 12 MPa and more preferably between 1 MPa and 10 MPa;
space velocity, defined as the ratio of the volume flow rate of the feedstock at ambient temperature and pressure to the volume of the catalyst, of between 0.1 h⁻¹ and 10 h⁻¹, preferably between 0.2 h⁻¹ and 7 h⁻¹, more preferentially between 0.5 h⁻¹ and 5 h⁻¹;
hydrogen flow rate of between 100 and 2000 normal liters of hydrogen per liter of feedstock per hour and preferably between 150 and 1500 normal liters of hydrogen per liter of feedstock and more preferentially between 300 and 1500 normal liters of hydrogen per liter of feedstock.

According to one or more embodiments, the hydrotreating and/or hydrocracking and/or hydroisomerization catalyst comprises at least one hydrogenating-dehydrogenating metal selected from the group comprising metals from group VIB and group VIIIB of the Periodic Table and at least one solid which is a Bronsted acid, i.e. a solid capable of releasing one or more protons, and optionally a binder.

According to one or more embodiments, the hydrotreating and/or hydrocracking and/or hydroisomerization catalyst comprises at least one group VIIIB noble metal chosen from ruthenium, rhodium, palladium, osmium, iridium and platinum, taken alone or as a mixture, and preferably from platinum and palladium, taken alone or as a mixture, and preferably used in their reduced form.

According to one or more embodiments, the hydrotreating and/or hydrocracking and/or hydroisomerization catalyst comprises: at least one metal chosen from nickel, molybdenum, tungsten, cobalt, ruthenium, indium, palladium and platinum; at least one support chosen from aluminas, boron oxides, magnesias, zirconias, titanium oxides and clays. According to one or more embodiments, the support is an alumina, silica-alumina, siliceous alumina or silica.

According to one or more embodiments, the hydrotreating and/or hydrocracking and/or hydroisomerization catalyst comprises at least one group VIIIB base metal selected from nickel and cobalt in combination with at least one group VIB metal selected from molybdenum and tungsten, used alone or as a mixture, and preferably used in their sulfide form.

According to one or more embodiments, in the case where said hydrotreating and/or hydrocracking and/or hydroisomerization catalyst comprises at least one group VIIIB noble metal, the content of noble metal in said catalyst is between 0.01% and 5% by weight, preferably between 0.05% and 4% by weight and very preferentially between 0.10% and 2% by weight, relative to the total weight of the catalyst.

According to one or more embodiments, in the case where said hydrotreating and/or hydrocracking and/or hydroisomerization catalyst comprises at least one group VIB metal in combination with at least one group VIII non-noble metal chosen from nickel and cobalt, the content of group VIB metal in said catalyst is, as oxide equivalent, between 5% and 40% by weight, preferably between 10% and 35% by weight, and the content of group VIIIB metal in said catalyst is, as oxide equivalent, between 0.5% and 15% by weight, preferably between 1% and 10% by weight, preferably between 1% and 8% by weight, and very preferentially between 1.5% and 6% by weight, relative to the total weight of the catalyst.

According to one or more embodiments, the hydrotreating and/or hydrocracking and/or hydroisomerization catalyst comprises or consists of at least one noble metal and a support comprising or consisting of at least one zeolite and at least one binder.

According to one or more embodiments, the zeolite-based hydrotreating and/or hydrocracking and/or hydroisomerization catalyst is advantageously of bifunctional type, i.e. it possesses a hydrogenating-dehydrogenating function and a hydroisomerizing function.

### Third Separation Unit

In the third separation unit 10, the RWGS gas 9 is treated, for example by condensation, in order to produce the water-depleted RWGS gas 12 (compared to the RWGS gas 9) and to recycle for example the second water effluent 11 to the water electrolysis unit 5.

According to one or more embodiments, the water-depleted RWGS gas 12 comprises less than 1 mol % water, preferably less than 0.5 mol % water, very preferably less than 0.25 mol % water.

The water-depleted RWGS gas 12 is sent to the FT reaction unit 13.

### Turbine

With reference to FIG. 1, according to one or more embodiments, the present invention makes it possible to recover energy in the form of electricity by means of at least one turbine 26.

According to one or more embodiments, the first turbine 26 is suitable for treating at least one portion 24 of the carbon dioxide-depleted gaseous effluent 18 in order to generate electricity. According to one or more embodiments, a second turbine (not shown) is suitable for treating at least in part, the first steam 22 and/or the second steam 23 in order to produce electricity (not shown).

According to one or more embodiments, the electricity is used to supply heat energy to the RWGS reaction unit 8 and/or to the carbon dioxide capture unit 2 and/or to the water electrolysis unit 5. According to one or more embodiments, the electricity 25 is used to supply heat energy to the RWGS reaction unit 8. According to one or more embodiments, the electricity 25 can be used to power an electric furnace for preheating the feedstock of the RWGS reaction unit 8.

### Carbon Dioxide Separation Unit

According to one or more embodiments, the device further comprises a unit for separating carbon dioxide and optionally methane (not shown), compounds potentially present in the RWGS gas 9. Advantageously, the carbon dioxide can be recycled into the RWGS reaction unit 8.

According to one or more embodiments, the carbon dioxide separation unit is positioned between the RWGS reaction unit 8 and the FT reaction unit 13. Advantageously, the size of the FT reaction unit 13 can thus be reduced.

According to one or more embodiments, the carbon dioxide separation unit is disposed at the outlet of the FT reaction unit 13.

### Oxy-Fuel Combustion Unit

According to one or more embodiments, the oxygen 6 obtained from the water electrolysis unit is upgraded in an oxy-fuel combustion (partial or complete oxidation) unit, for example to convert the methane formed that is present in the RWGS gas 9 separated by the carbon dioxide separation unit.

### Effluent Purification Units

According to one or more embodiments, the carbon dioxide-rich effluent 3 and/or the carbon dioxide-rich gaseous effluent 35 are purified, separately or after mixing, before being introduced into the RWGS reaction unit 8. According to one or more embodiments, the RWGS gas 9 is purified before being introduced into the FT reaction unit 13, upstream or downstream of the third separation unit 10. According to one or more embodiments, the first water effluent 16 is purified before being introduced into the water electrolysis unit 5. The effluent purification steps aim to at least partially eliminate the sulfur-containing and nitrogen-containing compounds, halogens, heavy metals and transition metals. The main technologies for purifying the gases are: adsorption, absorption, catalytic reactions.

In the present patent application, the groups of chemical elements are given, by default, according to the CAS classification (CRC Handbook of Chemistry and Physics, published by CRC Press, Editor-in-Chief D. R. Lide, 81st edition, 2000-2001). For example, group VIIIB according to the CAS classification corresponds to the metals from columns 8, 9 and 10 according to the new IUPAC classification; group VIB according to the CAS classification corresponds to the metals from column 6 according to the new IUPAC classification.

### Example

The various examples relate to sequences, in accordance or not in accordance with the invention, the objective of which is to produce a hydrocarbon cut from flue gases containing 21% by weight of carbon dioxide. The flow rate of flue gases to be treated is 3641 kg/h for all the examples.

### Example 1 not in Accordance with the Invention

The example of a device not in accordance with the invention is similar to the device shown in FIG. 1, except that the second separation unit 34 is not present and that there is thus no carbon dioxide-rich gaseous effluent 35 directed toward the RWGS reaction unit 8.

This example illustrates the operation of the sequence with the upgrading of the portion 24 of the carbon dioxide-depleted gaseous effluent 18 from the first separation unit 15 with combustion step in order to generate heat for the RWGS reaction unit 8. The carbon dioxide present in the carbon dioxide-depleted gaseous effluent 18 is not separated before entering the air combustion reaction unit 28.

The flow rate of flue gases feeding the carbon dioxide capture unit 2 is 3641 kg/h, to which must be added the flow rate of flue gases of the combustion effluent 29, which gives a total flow rate of feedstock 1 of 5509 kg/h. The flow rate of the carbon dioxide-rich effluent 3 from the carbon dioxide capture unit 2 is 1283 kg/h, sent to the RWGS reaction unit 8.

1331 kg/h of water 4 supplies the water electrolysis unit 5 of which 672 kg/h is fresh water. The power consumption of the water electrolysis unit 5 is 5.8 MWe.

The amount of first steam 22 produced by the FT reaction unit 13 is 1310 kg/h. The heat exchanger 31 produces 1295 kg/h of second steam 23 out of the 2103 kg/h required for the operation of the carbon dioxide capture unit 2. The steam requirements of the reboiler of the carbon dioxide capture unit 2 are covered.

The production of hydrocarbon cut 21 is 177 kg/h.

Table 1 summarizes the flow rates at the inlet and outlet of the process units.

TABLE 1

| — | Inlet | | Outlet | | | | |
|---|---|---|---|---|---|---|---|
| — | 3 | 4 | 6 | 11 | 16 | 24 | 21 |
| Flow rate | 1283 | 1331 | 1208 | 358 | 301 | 571 | 177 |
| in kg/h | | | | | | | |

### Requirements:

consumption of the water electrolysis unit 5: 5.8 MWe;
heat consumed by the RWGS reaction unit 8 at 864° C.: 0.2 MWth;
heat needed to preheat the feedstock (3+7) at the inlet of unit 8 to 864° C.: 0.8 MWth;
steam to the reboiler of the carbon dioxide capture unit 2: 2103 kg/h.

### Energy Recovery:

heat released by the air combustion reaction unit 28 at 1200° C. (with 20% excess air): 0.35 MWth;
heat recovered during cooling of the flue gases at the outlet of the air combustion reaction unit 28 from 1200° C. to 150° C.: 0.7 MWth (to preheat the feedstock (3+7) at the inlet of unit 8);
steam produced at the heat exchanger 31: 1295 kg/h;
steam produced in the FT reaction unit 13: 1310 kg/h;
electricity production at the first turbine 26: 5 kWe.

### Example 2 in Accordance with FIG. 1

Example 2 is in accordance with the invention according to FIG. 1. This example illustrates the operation of the sequence with the upgrading of the portion 24 of the carbon dioxide-depleted gaseous effluent 18 to an air combustion unit in order to generate heat for the RWGS reaction unit 8. The carbon dioxide-depleted gaseous effluent 18 is obtained from the second separation unit 34, making it possible to remove a portion of the carbon dioxide from the effluent 33 obtained from the first separation unit 15. The carbon dioxide-rich gaseous effluent 35 is sent to the RWGS reaction unit 8. The portion 24 of the carbon dioxide-depleted gaseous effluent 18 is directed toward the combustion unit.

The flow rate of flue gases feeding the carbon dioxide capture unit 2 is 3641 kg/h, to which must be added the flow rate of flue gases of the combustion effluent 29, which gives a total flow rate of feedstock 1 of 5332 kg/h. The flow of carbon dioxide-rich effluent 3 from the carbon dioxide capture unit 2 is mixed with the carbon dioxide-rich gaseous effluent 35, and their total flow rate amounts to 1352 kg/h sent to the RWGS reaction unit 8.

1403 kg/h of water 4 supplies the water electrolysis unit 5 of which 708 kg/h is fresh water. The power consumption of the water electrolysis unit 5 is 6.6 MWe.

The amount of first steam 22 produced by the FT reaction unit 13 is 1377 kg/h. The heat exchanger 31 produces 1332 kg/h of second steam 23 out of the 2392 kg/h required for the operation of the carbon dioxide capture unit 2 and the second separation unit 34. The steam requirements of the reboiler of the carbon dioxide capture unit 2 and of the second separation unit 34 are covered.

With the same amount of treated flue gases as in example 1, the production of hydrocarbon cut 21 is 186 kg/h instead of the 177 kg/h previously.

Table 2 summarizes the flow rates at the inlet and outlet of the process units.

TABLE 2

| — | Inlet | | Outlet | | | | |
|---|---|---|---|---|---|---|---|
| — | 3 + 35 | 4 | 6 | 11 | 16 | 24 | 21 |
| Flow rate | 1283 | 1331 | 1208 | 358 | 301 | 571 | 177 |
| in kg/h | | | | | | | |

### Requirements:

consumption of the water electrolysis unit 5: 6.6 MWe;
heat consumed by the RWGS reaction unit 8 at 864° C.: 0.2 MWth;
heat needed to preheat the feedstock (3+35+7) at the inlet of unit 8 to 864° C.: 0.8 MWth;
steam to the reboiler of the carbon dioxide capture unit 2: 2002 kg/h.
steam to the reboiler of the second separation unit 34: 390 kg/h.

### Energy Recovery:

heat released by the air combustion reaction unit 28 at 1200° C. (with 20% excess air): 0.56 MWth;
heat recovered during cooling of the flue gases at the outlet of the air combustion reaction unit 28 from 1200° C. to 150° C.: 0.7 MWth (to preheat the feedstock (3+35+7) at the inlet of unit 8);
steam produced at the heat exchanger 31: 1332 kg/h;
steam produced in the FT reaction unit 13: 1377 kg/h;
electricity production at the first turbine 26: 3.5 kWe.

1. A device for capturing and converting a carbon dioxide-containing feedstock, comprising the following units:
a unit (2) for capturing carbon dioxide from the feedstock (1) suitable for producing a carbon dioxide-rich effluent (3);
a water electrolysis unit (5) suitable for converting water (4) in order to produce oxygen (6) and hydrogen (7);
a reverse water gas shift RWGS reaction unit (8) suitable for treating the carbon dioxide-rich effluent (3) with hydrogen (7) and producing an RWGS gas (9) enriched in carbon monoxide and water;
a Fischer-Tropsch reaction unit (13) suitable for converting the RWGS gas (9) and producing an FT effluent (14);
a first separation unit (15) suitable for treating at least a portion of the FT effluent (14) and producing a hydrocarbon effluent (17), a first water effluent (16), and a first gaseous effluent (33);
a second separation unit (34) suitable for treating the first gaseous effluent (33) and producing a carbon dioxide-depleted gaseous effluent (18) and at least partially sending a carbon dioxide-rich gaseous effluent (35) to the RWGS reaction unit (8); and
a hydrogen reaction unit (20) suitable for treating the hydrocarbon effluent (17) and producing at least one hydrocarbon cut (21).

2. The device as claimed in claim 1, comprising an air combustion reaction unit (28) suitable for at least partially oxidizing the carbon dioxide-depleted gaseous effluent (18), producing a combustion effluent (29) comprising carbon dioxide and water, and sending the combustion effluent (29) to the carbon dioxide capture unit (2).

3. The device as claimed in claim 1, wherein the air combustion reaction unit (28) is suitable for producing heat used to provide heat energy to the RWGS reaction unit (8) and/or the carbon dioxide capture unit (2).

4. The device as claimed in claim 2, wherein the air combustion reaction unit (28) is suitable for producing heat used to provide heat energy to the RWGS reaction unit (8).

5. The device as claimed in claim 2, wherein the air combustion reaction unit (28) is suitable for heating the carbon dioxide-rich effluent (3) and/or the carbon dioxide-rich gaseous effluent (35) and/or the hydrogen (7), or integrating the reaction section of the RWGS reaction unit (8) into a combustion chamber.

6. The device as claimed in claim 1, wherein the Fischer-Tropsch reaction unit (13) is suitable for generating a first steam (22) for supplying thermal energy to the carbon dioxide capture unit (2).

7. The device as claimed in claim 1, comprising a first heat exchanger (31) suitable for generating a second steam (23) by heat exchange between water and the RWGS gas (9).

8. The device as claimed in claim 1, comprising a first turbine (26) for at least partially treating the carbon dioxide-depleted gaseous effluent (18) in order to generate electricity.

9. The device as claimed in claim 8, wherein electricity is used to supply heat energy to the RWGS reaction unit (8) and/or the carbon dioxide capture unit (2) and/or the water electrolysis unit (5).

10. The device as claimed in claim 8, wherein the water electrolysis unit (5) treats water from a makeup line and/or RWGS gas (9) and/or FT effluent (14).

11. The device as claimed in claim 8, wherein the water from the RWGS gas (9) is partly or completely separated by a third separation unit (10) in order to be sent to the water electrolysis unit (5).

12. The device as claimed in claim 8, comprising a carbon dioxide separation unit positioned between the RWGS reaction unit (8) and the Fischer-Tropsch reaction unit (13).

13. A process for capturing and converting carbon dioxide, comprising the following steps:
treating the feedstock (1) in a carbon dioxide capture unit (2) in order to produce a carbon dioxide-rich effluent (3);
converting water (4) in a water electrolysis unit (5) in order to produce oxygen (6) and hydrogen (7);
treating the carbon dioxide-rich effluent (3) with the hydrogen (7) in a reverse water gas shift RWGS reaction unit (8), in order to produce an RWGS gas (9) enriched in CO and in water;
converting the RWGS gas (9) in a Fischer-Tropsch reaction unit (13) in order to produce an FT effluent (14);
treating the FT effluent (14) in a first separation unit (15) in order to produce at least one hydrocarbon effluent (17), a first water effluent (16) and a first gaseous effluent (33);
separating the first gaseous effluent (33) in a second separation unit (34) in order to produce a carbon dioxide-rich gaseous effluent (35) and a carbon dioxide-depleted gaseous effluent (18);
at least partially sending the carbon dioxide-rich gaseous effluent (35) to the RWGS reaction unit (8); and
treating the hydrocarbon effluent (17) in a hydrogen reaction unit (20) in order to produce at least one hydrocarbon cut (21).

14. The process as claimed in claim 13, wherein the RWGS reaction unit (8) comprises at least one reactor used under at least one of the following operating conditions:
temperature of between 700° C. and 1200° C.;
pressure of between 0.1 MPa and 10 MPa;
space velocity of the gas at the inlet of the reactor of between 5000 NL/kg_{cata}/h and 40 000 NL/kg_{cata}/h;
catalyst comprising at least one metal selected from the group consisting of the elements Ni, Cu, Fe, Co, Pt, Pd, Ru, Ag and Au, and/or
wherein the FT reaction unit (13) comprises at least one reactor used under at least one of the following operating conditions:
temperature of between 170° C. and 280° C.;
absolute pressure of between 1.0 MPa and 6.0 MPa;
catalyst comprising cobalt or iron.

15. The process as claimed claim 13, wherein the air combustion reaction unit (28) comprises at least one reactor used under at least one of the following operating conditions:
absolute pressure of between 0.1 MPa and 4 MPa;
temperature of between 600° C. and 2000° C.;
presence of air used for combustion, with an aeration rate of between 1 and 2, the aeration rate being defined as the ratio of the molar flow rate of air injected to the theoretical air flow rate for complete oxidation of all the fuels.
